(19) United States
(12) Patent Application Publication
(10) Pub. No.: US 2021/0369887 A1
(43) Pub. Date: Dec. 2, 2021

(54) SANITIZATION DEVICES

(71) Applicant: Hewlett-Packard Development Company, L.P., Spring, TX (US)

(72) Inventors: Dimitre Mehandjiysky, Spring, TX (US); Syed S. Azam, Spring, TX (US); John W. Frederick, Spring, TX (US); Jonathan Neuneker, Boise, ID (US); Ian Christopher Shatto, Boise, ID (US); Paul Michael Carson, Boise, ID (US)

(73) Assignee: Hewlett-Packard Development Company, L.P., Spring, TX (US)

(21) Appl. No.: 17/042,165

(22) PCT Filed: Jul. 31, 2018
(86) PCT No.: PCT/US2018/044539

§ 371 Date: Sep. 27, 2020

(51) Int. Cl. A61L 2/10; A61L 2/14

(52) U.S. Cl. CPC A61L 2/10; A61L 2/14; A61L 2202/20; A61L 2202/14; A61L 2202/11

(57) ABSTRACT

In some examples, a sanitization device includes an elongated member, a plurality of sanitizing sources located on a surface of the elongated member, and an attachment mechanism coupled to the elongated member, where the attachment mechanism is to couple the elongated member to an electronic device including a display.

### BACKGROUND

Equipment may become dirty over time and/or with use of the equipment. For instance, equipment such as electronic device may become dirty due to a user contacting a surface (e.g., a touchscreen or other type of display) of the electronic device. Some sanitizing approaches may utilize liquid such as alcohol, detergents, etc., in an effort to disinfect or otherwise clean the equipment.

### BRIEF DESCRIPTION OF THE DRAWINGS

FIG. 1 is an example of a sanitization device according to the present disclosure.

FIG. 2 is yet another example of a sanitization device according to the present disclosure.

FIG. 3A is yet another example of a sanitization device according to the present disclosure.

FIG. 3B is yet another example of a sanitization device according to the present disclosure.

FIG. 4 is an example of a sanitizing system according to the present disclosure.

FIG. 5 is yet another example of a sanitizing system according to the present disclosure.

FIG. 6 is yet another example of a sanitizing system according to the present disclosure.

FIG. 7 is an example of the actuation of an example of a sanitizing system according to the present disclosure.

FIG. 8 is yet another example of the actuation of an example of a sanitizing system according to the present disclosure.

FIG. 9 is an example of a controller consistent with the present disclosure.

FIG. 10 illustrates an example of a method consistent with the disclosure.

### DETAILED DESCRIPTION

As mentioned, an electronic device may become dirty over time and/or with use of the electronic device. As used herein, the term dirty refers to having a visible element (such a stain and/or dust) and/or an invisible elements such pathogen on a surface. A “pathogen” may be any infectious agent that can cause disease, such as a virus, bacterium, prion, fungus, viroid and/or parasite. As such, sanitization or otherwise cleaning an electronic device may be desired. Some approaches may attempt to manually sanitize an electronic device. However, manually sanitizing a large number of electronic devices and/or displays, particularly on a daily basis, may be time-consuming and inefficient. Moreover, ensuring that the manual sanitizing is actually performed and/or effectively performed (sanitizing an entire surface) can be difficult to track in a manual sanitization approach. Additionally, manual sanitization approaches may interfere with the day to day operation of a business (e.g., downtime during a manual sanitization operation). Further, such manual sanitization processes may not permit the use of various sanitizing methodologies such as those employing ultra-violet (UV) light and/or cold plasma due to user health and safety risks.

Accordingly, the present disclosure is directed to sanitization devices, As detailed herein, sanitization devices refer to a device that can be coupled to an electronic device and emit UV light and/or cold plasma to sanitize the electronic device and/or display. As used herein, the term “sanitizing”, “sanitize”, and “sanitization” refer to the destruction of pathogens and/or other kinds of microorganisms. For instance, in some examples, a sanitization device can translate in a pattern relative to an electronic device including a display to sanitize a surface of the electronic device and/or display with a UV light and/or cold plasma emission. Additionally, the sanitization device can be automated to enable users to personalize the start time of sanitizing and duration of the sanitation process. Notably, sanitization devices as detailed herein provide safe use of effective UV light and/or cold plasma sanitizing, consistent sanitation across the entire exterior of the electronic device and/or the display, and/or allow for scheduled sanitizing times.

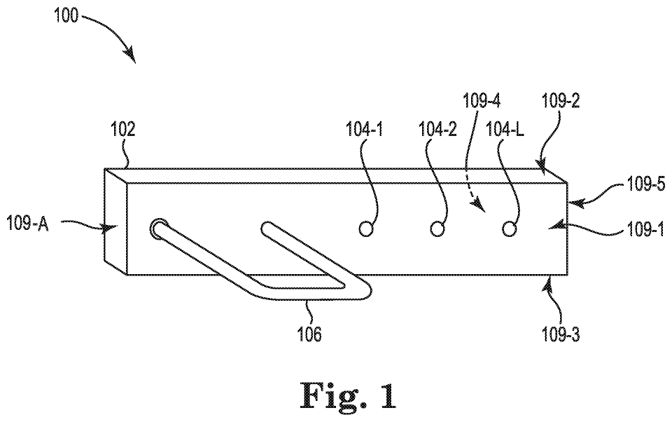

FIG. 1 is an example of a sanitization device 100 according to the present disclosure. As illustrated in FIG. 1, sanitization device 100 comprises an elongated member 102. The elongated member 102 can be composed of a material such as a plastic, a metal, a rubber or other elastomer, among other possibilities. Although FIG. 1 illustrates the elongated member as a flat bar, the elongated member can be composed in different forms such as being cylindrical, spherical, curved, poly-prismatic among other possibilities.

The elongated member 102 can contain a plurality of sanitizing sources 104-1, 104-2, . . . , 104-L (collectively referred to herein as sanitizing sources 104) across a plurality of surfaces 109-1, 109-2, 109-3, 109-4, 109-5, . . . , 109-A (collectively referred to herein as surfaces 109) of the elongated member 102. The plurality of sanitizing sources 104 can be located on a plurality of surfaces 109, an individual surface, or a combination of surfaces. For example, the plurality of sanitizing sources 104 can be located on a first surface 109-1, as illustrated in FIG. 1. However, the disclosure is not so limited, For instance, the sanitizing sources can be located on at least three of the first surface 109-1, a second surface 109-2, a third surface 109-3, a fourth surface 109-4, a fifth surface 109-5, and/or a sixth surface 109-A. Stated differently, the plurality of sanitizing sources 104 can be located on some or all exterior surfaces of the sanitization device 100. Further, it is noted that a total number of the exterior surfaces of the sanitization device 100 can be varied to include more or less exterior surfaces, for instance depending upon a shape of the sanitization device 100.

The plurality of sanitizing sources 104 can include sanitizing sources emitting UV light, cold plasma, detergents, heat, air, liquids, and/or any combination thereof, among other possibilities. For instance, in some examples, the sanitizing sources 104 can be UV light sanitizing sources and/or cold plasma emitting sanitizing sources 104. Although not shown, the plurality of sanitizing sources 104 can include a sanitizing source (not illustrated) located on the attachment mechanism 106.

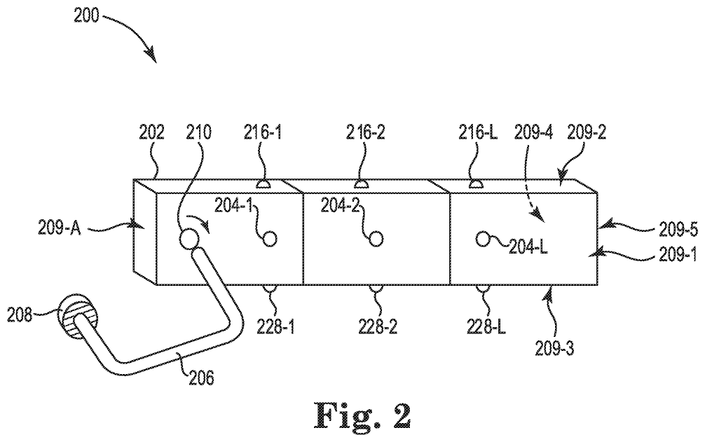

The elongated member 102 also can include an attachment mechanism 106. The attachment mechanism 106 can be coupled to the elongated member 102 and/or coupled to a motor (e.g., as described herein with respect to FIG. 2). The attachment mechanism 106 can movably couple the elongated member to a surface of an electronic device. A surface can be defined as, but not limited to, an outside part or uppermost layer of an object. As used herein, being “movably coupled” refers to being permantly or removably coupled to a surface of an electronic device in a manner to permit movement of the elongated member relative to a display of the electronic display.

The attachment mechanism 106 can be either removably coupled or permanently coupled to the elongated member 102. Similarly, the attachment mechanism 106 can also be removably coupled or permanently coupled to various other devices such as a motor, as described herein. The attachment mechanism 106 is to removably or permanently couple the elongated member to a surface of an electronic device. As used herein, removably coupled refers to coupling elements in a manner that the elements are intended to be decoupled, for instance decoupling (with a tool or otherwise in an intended manner) without breaking an element. As used herein, permanently coupled refers to coupling elements in a manner that the elements are not intended to be decoupled, for instance decoupling (with a tool or otherwise) physically breaks an element. Although the attachment mechanism 106 is shown in FIG. 1 as U-shaped in form, it can incorporate other forms such as cylindrical, straight, curved, spherical, among other possibilities.

Additionally, although an individual attachment mechanism 106 is shown, there can also be a plurality of attachment mechanisms. The attachment mechanism 106 can also be formed of a flexible material, a rigid material, or a combination thereof. The attachment mechanism 106 can also be located on any of the plurality of surfaces 109 inclusive of the first surface 109-1, a second surface 109-2, a third surface 109-3, a fourth surface 109-4, a fifth surface 109-5, a sixth surface 109-A or any combination thereof.

FIG. 2 is yet another example of a sanitization device 200 according to the present disclosure. The sanitization device 200 can be analogous to or similar to the sanitization device 100. For instance, the attachment mechanism 206 can be coupled (e.g., removably coupled as represented by element 210) to the elongated member 202 and can be actuated by a motor 208 or otherwise actuated.

The motor 208 can be removably or permanently coupled to the attachment mechanism 206. The motor 208 can be coupled to the elongated member to actuate the elongated member, relative to a surface, when the sanitization device 200 is coupled to the surface. In some examples, the motor 208 can be an electric motor. As used herein, the term “electric motor” refers to an electrical device that converts electrical energy into mechanical energy. The motor can actuate a sanitization device such as sanitization device 200 relative to an electronic device (not illustrated). For example, the motor 208 can contain the driving function to provide actuation of the elongated member 202, alternatively, the motor 208 can also not contain the driving function to provide actuation of the elongated member but rather act as a sliding or static device, among other possibilities. In some examples, the motor 208 can couple the attachment mechanism 206 and/or the elongated member 202 to an electronic device.

The elongated member 202 can include a plurality of sanitizing sources 204-1, 204-2, . . . , 204-L. For example, the elongated member 202 can include sanitization sources 204-1, 204-2, . . . , 204-L (collectively referred to as sanitization sources 204) on a first surface 209-1 of the elongated member. Similarly, the elongated member 202 can include a plurality of sanitization sources 216-1, 216-2, . . . , 216-L (collectively referred to as sanitization sources 216) on a second surface 209-2 of the elongated member.

Similarly, the elongated member 202 can include a plurality of sanitization sources 228-1, 228-2, . . . , 228-L (collectively referred to as sanitization sources 228) on a third surface 209-3 of the elongated member. Similarly, the elongated member 202 can include a plurality of sanitization sources (not illustrated for ease of illustration) on a fourth surface 209-4 of the elongated member, In some examples, the elongated member 202 can include a sanitization source (not illustrated) on a fifth surface 209-5 and/or a sixth surface 209-A. Additionally, any number and combination of sanitizing sources 204, 216, 228 can be located on the respective surfaces 209. For example, it is possible to have no sanitizing sources on the fourth surface 209-4 while having all other sanitizing sources 204, 216, 228 on the first surface 209-1, second surface 209-2, and third surface 209-3, respectively. Further, the plurality of sanitizing sources 204, 216, and/or 228 can operate simultaneously, individually, or in various combinations as a group. For example, sanitizing source 204-1 and 204-2 could be operating in conjunction with sanitizing source 216-2 to emit a sanitizing emission, while all other sanitizing sources (i.e. 228-1, 216-1, etc.) can be off (i.e. not operating), among other possibilities.

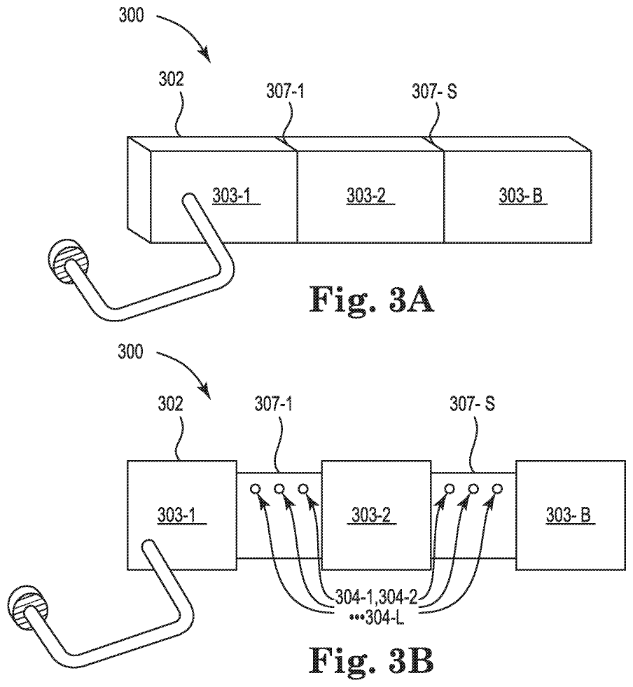

FIG. 3A is yet another example of a sanitization device 300 according to the present disclosure. The sanitization device 300 can be analogous or similar to sanitization device 100 and/or 200, as detailed at FIGS. 1 and 2, respectively. As illustrated in FIG. 3, the elongated member 302 can be comprised of different portions such as a first portion 303-1, second portion 303-2, and third portion 303-B (collectively referred to herein as portions 303). Although three portions 303 are shown in FIG. 3, it is noted that the elongated member 302 can have any number of portions 303. FIG. 3A represents a sanitization device 300 in a non-extended state, meaning the portions 303 are not extended and separated.

FIG. 3B is yet another example of a sanitization device 300 according to the present disclosure. As illustrated in FIG. 3B, the sanitization device 302 is in an extended state, meaning that a portion of a plurality of the portions 303 are separated from each other. The elongated member 302 is formed of a plurality of telescoping sections (307-1, . . . , 307-S) (collectively referred to herein as telescoping sections 307). The telescoping sections 307 can be located between the portion 303. For instance, as illustrated in FIG. 3, the telescoping sections 307 can be located between each of the sections 303, Although two telescoping sections 307 are shown, it is noted that the elongated member 302 can have any number of telescoping sections 307. In any case, the telescoping sections 307 can permit extension and retraction of the individual portions of the portions 303 relative to each other.

In some examples, each of the portions 303 can be the same in volumetric size, allowing for retraction up until point of contact of each portion, as illustrated in FIG. 3. However, in some examples each of the portions 303 can be different in volumetric size and/or shape. Telescoping sections 307 can be actuated manually or automatically (e.g., by an electric motor) to retract and/or telescope. In some examples, the telescoping sections 307 can be fixed (i.e. locked in place) or unfixed, when extended and/or retracted. In some examples, when extended, the telescoping sections 307 can be extended from each other to exposed surface a plurality of sanitizing sources, such as sanitizing sources 304-1, 304-2, . . . , 304-L.

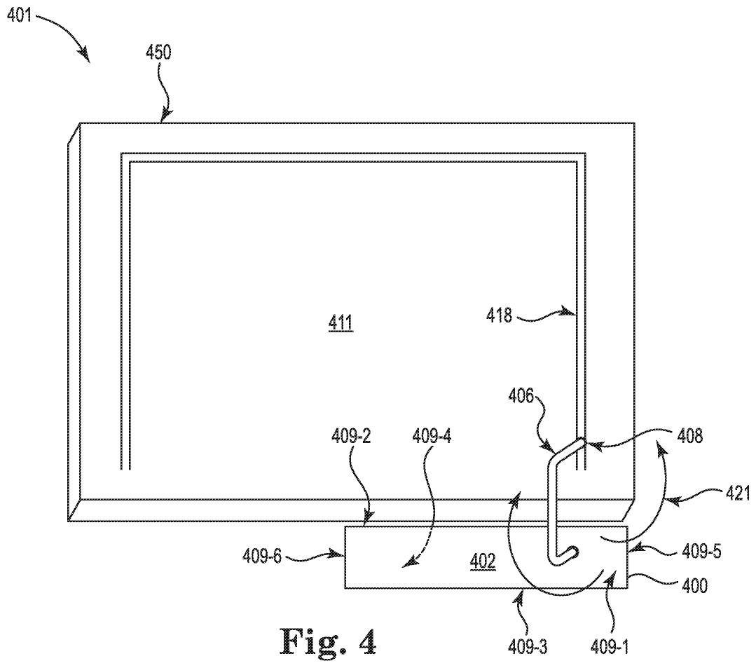

In various examples, a sanitizing device can sanitize surfaces of an electronic device such as a front and/or back surface of an electronic device. For instance, FIG. 4 is an example of a sanitizing system 401 according to the present disclosure. As illustrated in FIG. 4, the sanitizing system 401 comprises an electronic device450 and the sanitization device 400, including an elongated member 402. Further, as illustrated in FIG. 4, the elongated member can be in a first position 421 and attached to a back surface 411. For instance, the elongated member can be attached to the back surface via a motor 408 and/or attachment mechanism 406 at the motor pathway 418,

The electronic device 450 can be a laptop, a printer, a table, a monitor, a phone, among other possibilities. For instance, as detailed herein, the electronic device can include a display (not shown in FIG. 4 for ease of illustration). As used herein, the term “display” can, for example, refer to a device which can provide information to a user and/or receive information from a user. For instance, a display can include a graphical user interface (GUI) that can provide and/or receive information to and/or from a user. In some examples, the display can be touch enabled to permit interaction with the display via touch such as be an object (e.g., a stylus) and/or a digit of a user.

As illustrated in FIG. 4, the motor pathway 418 can be located on the back surface 411. Though, the motor pathway can be located on a different surface such as a side, top, and/or bottom surface of an electronic device. The motor pathway can be a channel, protrusion, track, belt, among other possibilities, The motor pathway 418 can couple the motor 408 to the back surface 411 and allow the motor to traverse in a sanitizing pattern. The motor pathway 418 can include various components such as magnets, gears, ridges, rubber, among other possibilities. The motor pathway 418 can be integrated directly in the back surface 411 or can be an external attachment of the display 450 allowing the motor pathway 418 to be removably coupled to the back surface 411, among other locations on a surface (side, front, etc.) of the electronic device 450. In some examples, actuation of the motor pathway can occur in place of or in combination with actuation of the motor. In such examples, the actuation would occur in the motor pathway 418 to move the motor and the sanitization device 400 around the electronic device in a sanitizing pattern, as described herein.

While FIG. 4 illustrate an elongated member extending across some but not all of a surface of an electronic device it is understood that the elongated member can extend across an entirety of a surface, in some examples. For instance, a position of a motor pathway can be varied (e.g., to a side surface of an electronic device) and an elongated member can extend from the motor pathway (e.g., to another motor pathway or different location of the motor pathway) or otherwise across an entirety of a surface such as a back and/or front surface.

As illustrated in FIG. 4, the sanitization device 400 is shown in the first position 421 beneath the electronic device 450. The sanitization device 400 can be analogous to or similar to the sanitization device 100, 200, and 300. Although not shown in FIG. 4 for ease of illustration, the first position 421 of the sanitization device 400 can permit the sanitization device 400 to be located in a plurality of locations around the sides of the electronic device 450 and/or directly behind the back surface 411 (so that it is not visible from the front of the electronic device 450). The sanitization device 400, can have its surfaces 409-1, 409-2, 409-3, 409-4, 409-5, 409-6 (collectively referred to herein as surfaces 409) oriented in different directions relative to the electronic device in different positions including those described herein. While an individual sanitization device 400 is shown, there can be a plurality of sanitization devices 400 along the electronic device 450.

The first position 421 is indicative of a stored position, though other stored positions are possible. While in a stored position, the sanitization device 400 is positioned to not obscure the display and/or use of any peripherals by a user. While in this position, the sanitization device can be off, meaning that the sanitizing sources are not active. In some examples, however, the sanitization device 400 can still be operating during a transition between positions further described below, meaning the sanitizing sources can be active. When the sanitizing sources are active in the first position 421, the sanitization device can sanitatize a stand of the electronic device 450 (not shown in the figure for ease), a bottom side (a surface adjacent surface 409-2 of the sanitization device as illustrated in FIG. 4) of the electronic device 450, and/or other peripheral devices such as keyboards, mice, and other peripheral devices (not shown).

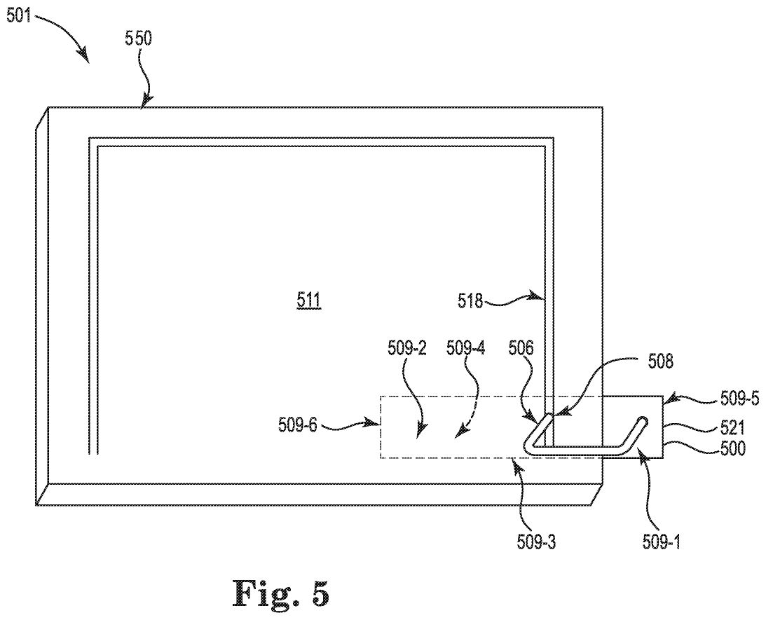

FIG. 5 is yet another example of a sanitizing system 501 according to the present disclosure. The sanitizing system 501 can be analogous to or similar to the sanitizing system 401 as described with respect to FIG. 4 herein. For instance, sanitizing system can include electronic device 550, motor pathway 518, back surface 511, surfaces 509-1, 509-2, 509-3, 509-4, 509-5, 509-6 and sanitization device 500, among other elements described above. As illustrated in FIG. 5, the sanitization device 500 can be in a second position 521 where the orientation of the first surface 509-1 of the sanitization device 500 is facing the front side of the electronic device 550 where display can be located.

The second position 521 can be achieved through the physical transition of the sanitization device from the first position (e.g., 421 as illustrated in FIG. 4) to the second position 521. This physical transition of the sanitization device from a first position to the second position 521 can be performed by a motor such as those described herein. Similarly, a motor can move the sanitization device to a different position such as a third position, as described herein. Additionally, the above described mechanisms can also be utilized in a similar fashion to move the sanitization device from a position (e.g., a third position) back to the first position.

The mechanism of the physical transition of the sanitization device from a first position to a second position 521 can involve the manipulation of the attachment mechanism 506 either manually or automatically (e.g., by a motor). Manipulation of the attachment mechanism can involve the act of rotating, pivoting, stretching, and/or bending of the attachment mechanism, among other possibilities to achieve proper transition.

In the second position 521, the sanitization device 500 can perform a sanitization operation, for instance to sanitize the front surface of the electronic device 550 utilizing active sanitizing sources. In some examples, the sanitizing sources of the sanitization device 500 can be inactive responsive to a user presence and/or occurrence of time of day, and among other possibilities.

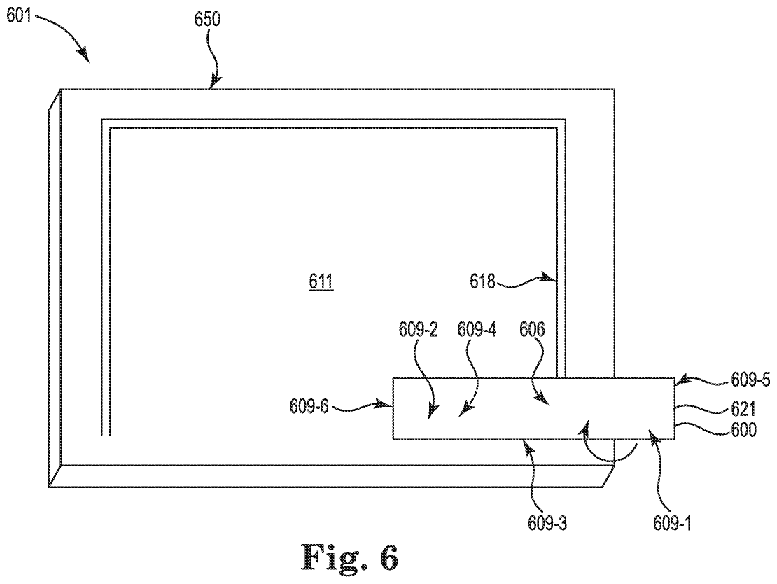

FIG. 6 is yet another example of a sanitizing system 601 according to the present disclosure. The sanitizing system 601 can be analogous to or similar to the sanitizing system 401 and/or 501. For instance, sanitizing system can include an electronic device 650, motor pathway 618, back surface 611, surfaces 609-1, 609-2, 609-3, 609-4, 609-5, 609-6 and sanitization device 600, among other relevant defined terms described above. In this figure, the sanitization device 600 is in a third position 621 where the orientation of the fourth surface 609-4 of the sanitization device 600 is facing the back surface 611.

In the third position 621, the sanitization device 600 is able to sanitize the back surface 611 utilizing active sanitizing sources. In some examples, the sanitizing sources of the sanitization device 611 can be inactive in the case of user presence, time of day, and among other possibilities.

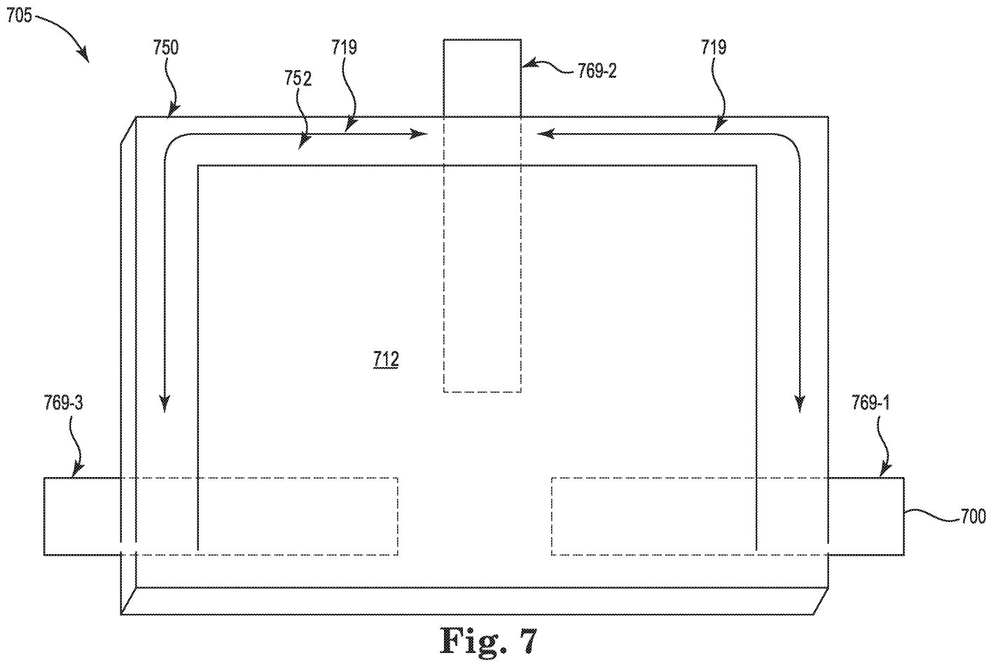

FIG. 7 is an example of the actuation of a sanitizing system 705 according to the present disclosure. As illustrated in FIG. 7 the sanitizing system 705 can include an electronic device 750 and the sanitization device 700, among other components. As illustrated in FIG. 7, the electronic device 750 can include a front surface 752 including a display 712 (e.g., a GUI). While the display 712 is illustrated on the front surface 752, in some examples a display can be include in a different surface of the electronic device such as the back and/or side surface of the electronic device,

A motor and/or a motor pathway, as described herein, can move the sanitization device 700 along a motor pathway in a sanitizing pattern 719 during a sanitation process. Sanitizing pattern 719 can allow for the movement of the sanitization device 700 relative to the electronic device 750 and/or the display 712. The sanitizing pattern 719 as shown in FIG. 7 can allow the sanitization device 700 to traverse between the sanitation positions 769-1, 769-2, and/or 769-3 any number of times. Sanitizing pattern 719 can involve straight patterns, circular patterns, zig-zag patterns, among other possible patterns. The sanitizing patterns 719 can be customized by a user or automated based on parameters (i.e. time of day, amount of sanitizing, including places on the electronic device such as the display which may be likely to be dirtier than other location on the electronic device, etc.).

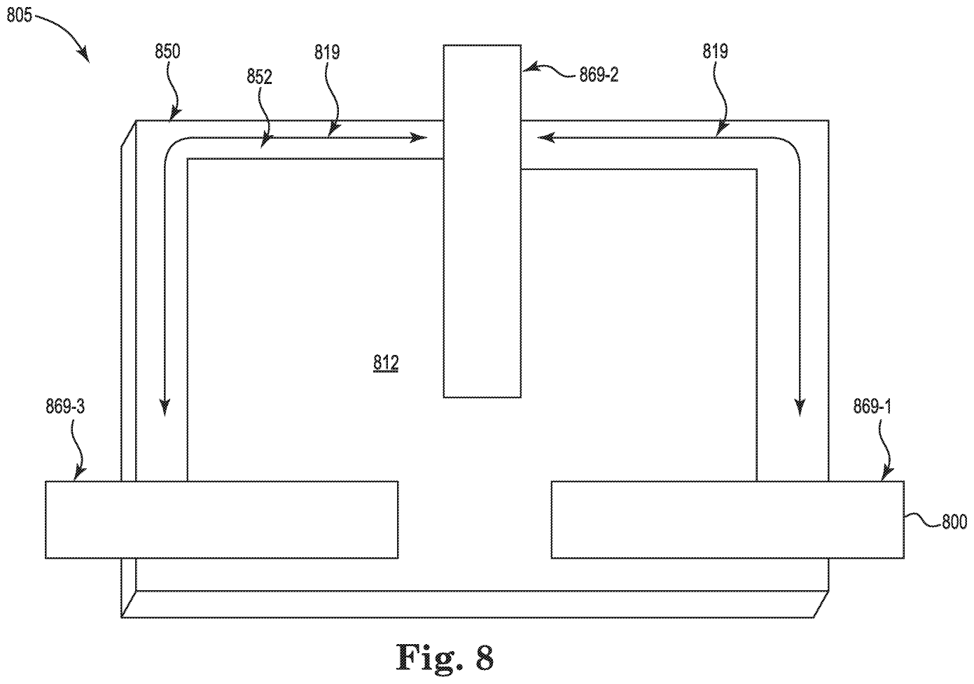

As illustrated in FIG. 7, the sanitization pattern 719 can permit sanitation of the back surface (not illustrated) of the electronic device 750, though sanitization of other surfaces of the electronic device and/or the display are possible. For instance, as illustrated in FIG. 8, the sanitization pattern can sanitize the front surface of the display. As used herein, the front surface refers to a surface of an electronic device that is located on the opposite side of an electronic device from the back surface (e.g., back surface 611 as illustrated in FIG. 6) of the electronic device 750. In some examples, the sanitization pattern 719 can permit sanitization of both the front surface and the back surface (and in some instances, additional surfaces such as a side surface and/or bezel of the electronic device).

FIG. 8 is yet another example of the actuation of an example of a sanitizing system 805 according to the present disclosure. As illustrated in FIG. 8, the sanitization system 805 can include an electronic device 850 and a sanitization device 800, among other components. As illustrated in FIG. 8, the system 805 can sanitize the front surface 852 such as the display 812. In some examples, the system 805 can sanitize a stand of the electronic device (not shown in the figure for ease of illustration). That is, the system 805 can include the same components as system 705 as described with respect to FIG. 7, however system 805 is shown as sanitizing the front surface 852 and the display 812 of the electronic device 850.

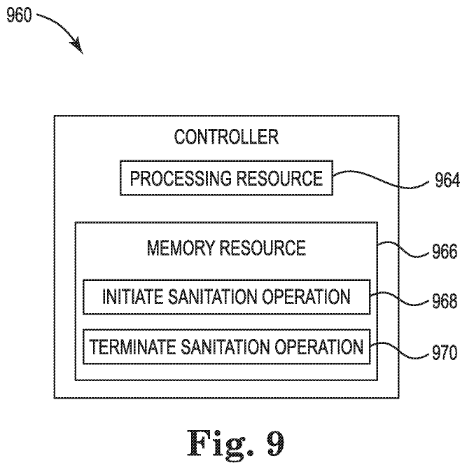

FIG. 9 is an example of a controller 960 consistent with the present disclosure. As described herein, the controller 960 can perform a function related to a sanitization devices. Although the following descriptions refer to an individual processing resource and an individual machine-readable storage medium, the descriptions can also apply to a system with multiple processing resources and multiple machine-readable storage mediums. In such examples, the controller 960 can be distributed across multiple machine-readable storage mediums and the controller 960 can be distributed across multiple processing resources. Put another way, the instructions executed by the controller 960 can be stored across multiple machine-readable storage mediums and executed across multiple processing resources, such as in a distributed or virtual computing environment.

As illustrated in FIG. 9, the controller 960 can comprise a processing resource 964, and a memory resource 966 storing machine-readable instructions 968, 970 to cause the processing resource 964 to perform an operation relating to a sanitization device, For instance, the controller 960 can include instructions to initiate and terminate a sanitation operation. As used herein, “cause” or “causing” refers to directly causing an action (e.g., asserting/de-asserting a signal sent to a motor and/or a sanitization device) or performing an action such as sending instructions to another component to cause the action.

Processing resource 964 can be a central processing unit (CPU), microprocessor, and/or other hardware device suitable for retrieval and execution of instructions stored in memory resource 966. Memory resource 966 can be a machine-readable storage medium can be any electronic, magnetic, optical, or other physical storage device that stores executable instructions. Thus, machine-readable storage medium can be, for example, Random Access Memory (RAM), an Electrically-Erasable Programmable Read-Only Memory (EEPROM), a storage drive, an optical disc, and the like. The executable instructions can be “installed” on a sanitization device and/or electronic device. Machine-readable storage medium can be a portable, external or remote storage medium, for example, that allows the sanitization device and/or electronic device (or a different device) to download the instructions from the portable/external/remote storage medium. In this situation, the executable instructions can be part of an “installation package”. As described herein, machine-readable storage medium can be encoded with executable instructions related to sanitization devices.

While FIG. 9 describes instructions 968, 970 with respect to the controller 960, some or all of the instructions 968, 970 can be stored and/or executed in a distributed computing environment such as in a cloud infrastructure that can manage or otherwise interact with sanitization device and/or electronic device.

The controller 960 can include instructions 968 stored in the memory resource 966 and executable by the processing resource 964 to cause the sanitization device to initiate a sanitization operation, as described herein. For instance, the sanitizing operation can be caused to initiate at a predetermined time of day, responsive to a user input, among other possibilities. For example, controller 960 can include instructions 968 stored in the memory resource 966 and executable by the processing resource 964 to cause a sanitization device to actuate relative to a surface of an electronic device and/or the display in a sanitizing pattern, described herein.

The controller 960 can include instructions 970 stored in the memory resource 966 and executable by the processing resource 964 to cause the sanitization device to terminate the sanitation operation. For instance, the instructions 970 can cause the sanitization operation to terminate (stop actuation of the sanitization device and/or return the sanitization device to a first position) responsive to a user input, reaching the end of a sanitization pattern, and/or at a given time of time (e.g., in advance of normal working hours, among other possibilities. As used herein, “terminate” or “initiate” refers to directly causing an action (e.g., asserting/de-asserting a signal sent to a motor and/or a sanitization device) or performing an action such as sending instructions to another component to cause the action such as initiation and/or termination of a sanitization operation.

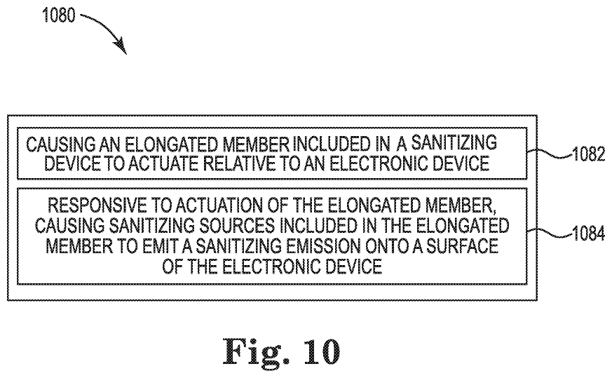

FIG. 10 illustrates an example of a method 1080 consistent with the disclosure. At 1082, the method 1080 can include causing an elongated member to actuate relative to an electronic device, as described herein. For instance, a controller can initiate a sanitization operation.

At 1084, the method 1080 can include causing sanitizing sources included in the elongated member to emit a sanitizing emission onto a surface of the electronic device, as described herein. For instance, the method can include responsive to actuation of the elongated member, causing sanitizing sources included in the elongated member to emit a sanitizing emission onto a surface of the electronic device.

In some examples, the method 1080 can include causing the actuation and sanitizing responsive to in response to an input. Examples of a suitable input include a user input, presence of a user, and/or a predetermined time of day. The user input can be provided to a sanitization device and/or to an electronic device such an electronic device coupled to a sanitization device.

In some examples, the method 1080 can include terminating a sanitization operation. For instance, as mentioned terminating (stop actuation of the sanitization device and/or return the sanitization device to a first position) can occur responsive to a user input, completion of the sanitization operation (e.g., reaching the end of a sanitization pattern), and/or at a given time of time (e.g., in advance of normal working hours, among other possibilities. In such examples, the method 1080 can further include causing the sanitization device to return to a stored position, as detailed herein, responsive to terminating the sanitization operation,

In some examples, the method 1080 can include causing the sanitizing sources to emit UV light, cold plasma, or a combination of UV light and cold plasma to remove bacteria, dirt, dust, fingerprints, condensation and other particulates from a surface of the electronic device.

The terms “including” and “having” are intended to have the same inclusive meaning as the term “comprising”. In the foregoing detailed description of the disclosure, reference is made to the accompanying drawings that form a part hereof, and in which is shown by way of illustration how examples of the disclosure can be practiced, These examples are described in sufficient detail to enable those of ordinary skill in the art to practice the examples of this disclosure, and it is to be understood that other examples can be utilized and that process, electrical, and/or structural changes can be made without departing from the scope of the disclosure.

The figures herein follow a numbering convention in which the first digit corresponds to the drawing figure number and the remaining digits identify an element or component in the drawing. Similar elements or components between different figures can be identified by the use of similar digits. For example, 100 can reference element “00” in FIG. 1, and a similar element can be referenced as 300 in FIG. 3. Elements shown in the various figures herein can be added, exchanged, and/or eliminated so as to provide a plurality of additional examples of the disclosure. In addition, the proportion and the relative scale of the elements provided in the figures are intended to illustrate the examples of the disclosure and should not be taken in a limiting sense.

What is claimed is:

1. A sanitization device comprising:
an elongated member;
a plurality of sanitizing sources located on a surface of the elongated member; and
an attachment mechanism coupled to the elongated member, wherein the attachment mechanism is to couple the elongated member to an electronic device including a display.

2. The device of claim 1, wherein the elongated member is formed of a plurality of telescoping sections.

3. The device of claim 1, wherein the sanitizing sources include UV light sources, cold plasma sources, or a combination of both UV light sources and cold plasma sources.

4. The device of claim 1, wherein the plurality of sanitizing sources are located on a plurality of surfaces of the elongated member, further comprising sanitizing sources located on at least three of a first surface, a second surface, a third surface, a fourth surface, a fifth surface, and a sixth surface of the elongated member.

5. The device of claim 1, wherein the attachment mechanism is to removably or permanently couple the elongated member to the electronic device.

6. The device of claim 5, wherein the elongated member is movably coupled via the attachment mechanism to the electronic device.

7. A sanitizing system comprising:
an electronic device including a display; and
a sanitization device comprising:
an elongated member:
a plurality of sanitizing sources located on a plurality of surfaces of the elongated member;
an attachment mechanism to couple the elongated member to an electronic device including a display; and
a motor coupled to the elongated member to actuate the elongated member relative to the display of the electronic device.

8. The system of claim 7, wherein the elongated member is comprised of a first portion, second portion, and third portion.

9. The system of claim 8, wherein the elongated member includes a first telescoping section between the first portion and the second portion and a second telescoping second between the second portion and third portion, and wherein the first telescoping section and the second telescoping section include a plurality of sanitization sources.

10. The system of claim 7, further comprising a controller to actuate the elongated member in a sanitizing pattern relative to the display.

11. The system of claim 7, wherein the electronic device is a laptop, table, monitor, a phone, or combinations thereof.

12. A sanitizing method comprising
causing an elongated member included in a sanitization device to actuate relative to an electronic device; and
responsive to actuation of the elongated member, causing sanitizing sources included in the elongated member to emit a sanitizing emission onto a surface of the electronic device.

13. The method of claim 12, further comprising causing the actuation and sanitizing responsive to in response to an input, wherein the input is the occurrence of a predetermined time of day or a user provided input.

14. The method of claim 12, further comprising causing the sanitization operation to terminate responsive to completion of the sanitation operation, and further comprising returning the sanitization device to a stored position responsive to completion of the sanitization operation.

15. The method of claim 12, further comprising instructions to cause the sanitizing sources to emit UV light, cold plasma, or a combination of UV light and cold plasma to remove bacteria, dirt, dust, fingerprints, condensation and other particulates from a surface of the electronic device.
